(19) United States
(12) Patent Application Publication
(10) Pub. No.: US 2008/0275440 A1
(43) Pub. Date: Nov. 6, 2008

(54) Post-ablation verification of lesion size

(75) Inventors: Paul S. Kratoska, Brooklyn Park, MN (US); Thomas R. Skwarek, Shoreview, MN (US)

Correspondence Address: SHUMAKER & SIEFFERT, P. A., 1625 RADIO DRIVE, SUITE 300, WOODBURY, MN, 55125, US

(73) Assignee: Medtronic, Inc.

(21) Appl. No.: 11/799,785

(22) Filed: May 3, 2007

(51) Int. Cl. A61B 18/18

(52) U.S. Cl. 606/41

(57) ABSTRACT

This disclosure is directed to a method of providing feedback regarding the outcome of ablation therapy. Measuring one or more tissue properties after the ablation procedure may allow the clinician to verify the size of the lesion formed or other therapy results. In one embodiment, the invention is directed toward a method for providing feedback regarding the results of tissue ablation, the method comprising deploying one or more needles from a catheter into a target tissue, delivering energy via at least one of the one or more needles to ablate at least a portion of the target tissue to form a lesion, stopping energy delivery via the at least one of the one or more needles, and measuring a tissue property via at least one of the one or more needles after the energy delivery has been stopped. The measured tissue property may be temperature or impedance. Also, the measured tissue property may be used to determine a volume of the lesion formed by ablation therapy.

### TECHNICAL FIELD

The invention relates to medical devices and, more particularly, to devices for controlling therapy delivery.

### BACKGROUND

Tissue ablation is a commonly used surgical technique to treat a variety of medical conditions, particularly when the treatment requires removing or destroying a target tissue. Medical conditions that can be treated by tissue ablation include, for example, benign prostatic hypertrophy, benign and malignant tumors, and destructive cardiac conductive pathways (such as ventricular tachycardia). Tissue ablation may also be used as part of common surgical procedures, for example, to remove or seal blood vessels.

Typically, ablation therapy involves heating the target tissue with a surgical instrument such as a needle or probe. The needle is coupled to an energy source that heats the needle, the target tissue, or both. Suitable energy sources include, for example, radio frequency (RF) energy, heated fluids, impedance heating, or any combination thereof.

Many ablation procedures are performed as minimally invasive procedures. Since the target tissue cannot be visually inspected during or after a minimally invasive treatment, the clinician usually selects therapy parameters (such as flow rate of conductive fluid, power delivered to the needle or probe, and treatment time) estimated to yield a preferred lesion size or other treatment result. The selected therapy parameters may be based on data collected from previous ablation procedures, the clinician's experience, and/or the condition of the patient.

### SUMMARY

In a minimally invasive procedure, a clinician cannot directly observe the results of the ablation therapy. Measuring one or more tissue properties after the ablation procedure may allow the clinician to verify the size of the lesion formed or other therapy results. In general, this disclosure is directed to methods for providing feedback on the outcome of ablation therapy.

In one embodiment, the invention is directed to a method for providing feedback regarding the results of tissue ablation, the method comprising deploying one or more needles from a catheter into a target tissue, delivering energy via at least one of the one or more needles to ablate at least a portion of the target tissue to form a lesion, stopping energy delivery via the at least one of the one or more needles, and measuring a tissue property via at least one of the one or more needles after the energy delivery has been stopped.

In another embodiment, the invention is directed to a system comprising a generator that generates energy to ablate at least a portion of a target tissue to form a lesion, one or more needles that deliver the energy to the target tissue, wherein at least one of the needles comprises a measurement device that measures a tissue property of the target tissue after the lesion is formed, and a processor that analyzes the measured tissue property and provides an indicator of the therapy outcome based on the measured tissue property.

In yet another embodiment, the invention is directed to a computer-readable medium comprising instructions for causing a programmable processor to deliver energy via one or more needles to ablate at least a portion of a target tissue to form a lesion, receive a tissue property measurement, wherein the tissue property measurement is measured via at least one of the one or more needles after the energy delivery has been stopped, and analyze the measured tissue property and provide an indicator of the therapy outcome based on the measured tissue property.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a conceptual diagram illustrating an example generator system in conjunction with a patient.

FIG. 2 is a side view of an example hand piece and connected catheter that delivers therapy to target tissue.

FIGS. 3A and 3B are cross-sectional side views of an example catheter tip in which a therapy needle exits to reach the target tissue.

FIGS. 4A and 4B are cross-sectional front views of an example catheter tip and exiting needles.

FIGS. 5A, 5B, 5C and 5D are cross-sectional front views of exemplary needles with varying sensing element configurations.

FIG. 6 is a functional block diagram illustrating components of an exemplary generator system.

FIG. 7 is a flow diagram illustrating an example technique for providing feedback regarding the outcome of ablation therapy.

### DETAILED DESCRIPTION

In a minimally invasive procedure, the clinician cannot directly observe the results of the ablation therapy. While power, time, and flow rate of conductive fluid (if used in the procedure) can be correlated with a specific lesion volume produced by the procedure, this correlation is only approximate. If the desired lesion is not successfully formed, the patient may continue to experience symptoms and additional ablation treatments may be necessary. This disclosure is directed to a method of providing feedback regarding the outcome of ablation therapy. Measuring one or more tissue properties after the ablation procedure may allow the clinician to verify the size of the lesion formed or other therapy results. For example, tissue impedance may be measured after the ablation procedure and measured impedance values may be used to determine the volume of the lesion formed.

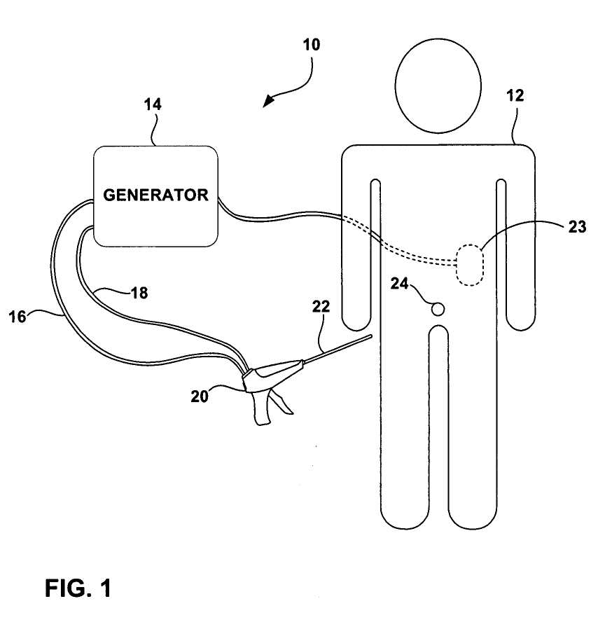

FIG. 1 is a conceptual diagram illustrating an example generator system in conjunction with a patient. As shown in the example of FIG. 1, system 10 may include a generator 14 that delivers therapy to treat a condition of patient 12, such as benign prostatic hypertrophy (BPH).

BPH is a condition caused by the second period of continued prostate gland growth. This growth begins after a man is approximately 25 years old and may begin to cause health problems after 40 years of age. The prostate growth eventually begins to constrict the urethra and may cause problems with urination and bladder functionality. Minimally invasive ablation therapy may be used to treat this condition. A catheter is inserted into the urethra of a patient and directed to the area of the urethra adjacent to the prostate. An ablation needle is extended from the catheter and into the prostate. The clinician performing the procedure selects the desired ablation parameters and the needle heats the prostatic tissue, which may be destroyed and later absorbed by the body. Ablation therapy shrinks the prostate to a smaller size that no longer interferes with normal urination and bladder functionality, and the patient may be relived of most problems related to BPH.

In the exemplary embodiment illustrated in FIG. 1, generator 14 is a radio frequency (RF) generator that provides RF energy to heat tissue of the prostate gland 24. This ablation of prostate tissue destroys a portion of the enlarged prostate caused by, for example, BPH. The RF energy is transmitted through electrical cable 16 to therapy device 20. The energy is then transmitted through a catheter 22 and is delivered to prostate 24 by a needle electrode (not shown in FIG. 1). A conductive fluid may be pumped out of generator 14, through tubing 18, into therapy device 20, and through catheter 22 to interact with the RF energy being delivered by the needle. This “wet electrode” may increase the effective heating area of the needle and increase therapy efficacy. Ground pad 23 may be placed at the lower back of patient 12 to return the energy emitted by the needle electrode.

The needle electrode that delivers energy to prostate 24 may also be used to measure a tissue property after ablation therapy is stopped. In other embodiments, a separate needle may be provided to measure the tissue property. Measuring a tissue property, such as tissue impedance or temperature, after the ablation therapy is stopped may help provide the clinician assurance that the ablation therapy was successful. Measured tissue property values may be used to confirm lesion formation and verify the size of the lesion formed.

In the illustrated example, generator 14 is an RF generator that includes circuitry for developing RF energy from an included rechargeable battery or a common electrical outlet. The RF energy is produced within parameters that are adjusted to provide appropriate prostate tissue heating. The RF current is conveyed from generator 14 via electrical cable 16 which is connected to the generator. The conductive fluid is provided to the needle by a pump (not shown) located within generator 14. In some embodiments, a conductive fluid may not be used in conjunction with the RF energy. This embodiment may be referred to as a “dry electrode” ablation system. Alternatively, other energy sources may be used in place of RF energy. Also, tissue property measurements may be used with both dry and wet ablation systems. With wet electrode ablation, there is potentially less feedback for the clinician than with dry electrode therapy, so tissue property measurements may be particularly useful with wet ablation therapy.

Therapy energy and other associated functions such as fluid flow are controlled via a graphic user interface located on a color liquid crystal display (LCD), or equivalent screen of generator 14. The screen may provide images created by the therapy software, and the user may interact with the software by touching the screen at certain locations indicated by the user interface. In this embodiment, no additional devices, such as a keyboard or pointer device, are needed to interact with the device. The touch screen may also enable device operation. In some embodiments, the device may require an access code or biometric authorization to use the device. Requiring the clinician to provide a fingerprint, for example, may limit unauthorized use of the system. Other embodiments of generator 14 may require input devices for control, or the generator may require manual operation or allow minimal computer control of the ablation therapy.

Cable 16 and tube 18 are connected to generator 14. Cable 16 conveys RF energy, and tube 18 conducts fluid from generator 14 to therapy device 20. Cable 16 may also include wiring coupled to a sensor (not shown) that detects a tissue property. In other embodiments, a separate cable may include this sensing wiring. Tube 18 may carry conductive fluid and/or cooling fluid to the target tissue, or an additional tube (not shown) may carry the cooling fluid used to irrigate the urethra of patient 12.

Therapy device 20 may be embodied as a hand-held device as shown in FIG. 1. Therapy device 20 may include a trigger to control the start and stop of therapy. The trigger may also deploy the needle into the target tissue. Attached to the distal end of therapy device 20 is a catheter 22. Catheter 22 may provide a conduit for both the RF energy and the fluid. Since catheter 22 enters patient 12 through the urethra, the catheter may be very thin in diameter and long enough to reach the prostate.

The end of catheter 22 may contain one or more electrodes for delivering RF current to the tissue of enlarged prostate 24. Catheter 22 may contain an ablation needle that acts as an electrode for penetrating into an area of prostate 24 from the urethra. More than one needle electrode may be used in system 10.

When RF energy is being delivered, the target tissue may increase in temperature, which destroys a certain volume of tissue. This heating may last a few seconds or a few minutes. A cooling fluid may be delivered to patient 12 via catheter 22 to help prevent damage to the urethra or other tissues proximate to prostate 24. For example, a cooling fluid may exit small holes in catheter 22 and flow around the urethra. In some embodiments, a conductive fluid may exit small holes in the needle and flow around the electrode. This conducting fluid, e.g., saline, may increase the effective heating area and decrease the heating time for effective treatment. Additionally, ablating tissue in this manner may enable the clinician to complete therapy by repositioning the needle a reduced number of times. In this manner, patient 12 may require fewer treatment sessions to effectively treat BPH.

In some cases, therapy device 20 may only be used for one patient. Reuse may cause infection and contamination, so it may be desirable for the therapy device to only be used once. A feature on therapy device 20 may be a “smart chip” in communication with generator 14. For example, when the therapy device is connected to generator 14, the generator may request use information from the therapy device. If the device has been used before, generator 14 may disable all functions of the therapy device to prevent reuse of the device. Once therapy device 20 has been used, the smart chip may create a use log to identify the therapy delivered and record that the device has been used. The log may include graphs of RF energy delivered to the patient, total RF energy delivered in terms of joules or time duration, error messages created, or any other information pertinent to the therapy.

In other embodiments, catheter 22 may independently include the needle such that different catheters may be attached to therapy device 20. Different catheters 20 may include different configurations of needles, such as lengths, diameters, number of needles, or sensors in the needles. In this manner, a clinician may select the desired catheter 22 that provides the most efficacious therapy to patient 12.

While the example of system 10 described herein is directed toward treating BPH in prostate 24, system 10 may be utilized at any other target tissue of patient 12. For example, the target tissue may be polyps in a colon, a kidney tumor, esophageal cancer, uterine cancer tissue, or liver tumors. In any case, a tissue property is detected after the ablation procedure to provide feedback regarding the outcome of the therapy. For example, tissue temperature and/or tissue impedance may be measured to estimate the volume of lesion formed.

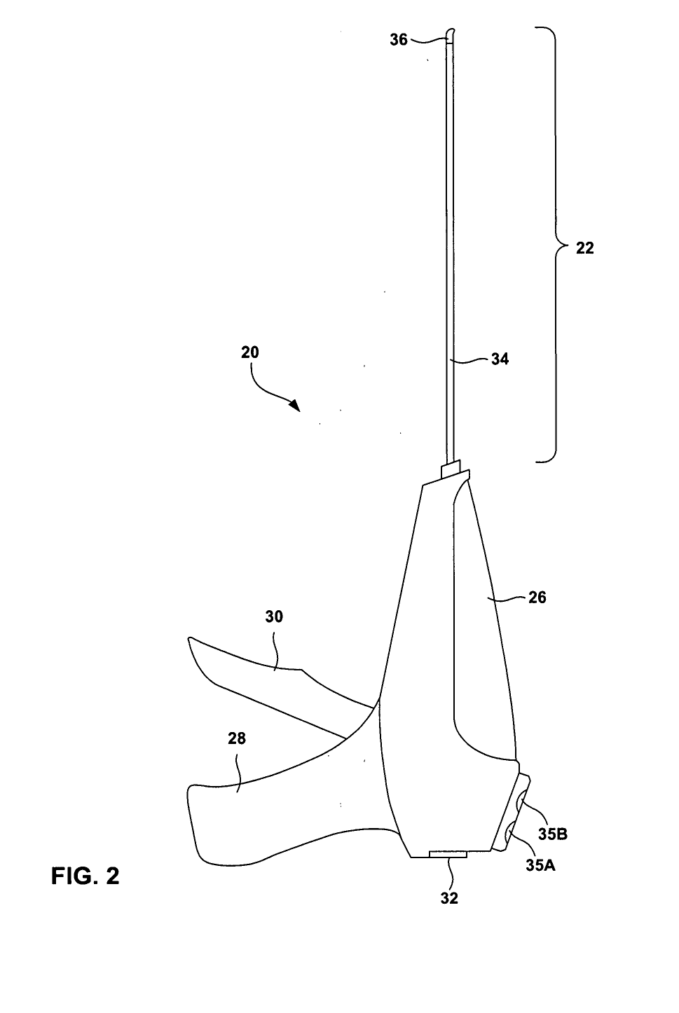

FIG. 2 is a side view of an example hand piece and connected catheter that delivers therapy to a target tissue. As shown in FIG. 2, therapy device 20 includes housing 26. Housing 26 includes ports 35A and 35B that may be used to couple cable 16 and tubing 18 (FIG. 1) to therapy device 20. Housing 26 is coupled to trigger 30 and includes handle 28. A cystoscope (not shown), may be inserted though axial channel 32 and fitted within catheter 22. Catheter 22 includes shaft 34 and tip 36. A clinician holds handle 28 and trigger 30 to guide catheter 22 through a urethra. The clinician may use the cystoscope to view the urethra through tip 36 and locate a prostate for positioning the needle (not shown) into prostate 24 from the tip 36. Once the clinician identifies correct placement for the needle, trigger 30 is squeezed toward handle 28 to extend the needle into prostate 24.

Housing 26, handle 28 of housing 26, and trigger 30 of therapy device 20 are constructed of a lightweight molded plastic such as polystyrene. In other embodiments, other injection molded plastics may be used such as polyurethane, polypropylene, high molecular weight polyurethane, polycarbonate or, nylon. Alternatively, construction materials may be aluminum, stainless steel, a metal alloy or a composite material. In addition, housing 26, handle 28 of housing 26, and trigger 30 may be constructed of different materials instead of being constructed out of the same material. In some embodiments, housing 26, handle 28 of housing 26, and trigger 30 may be assembled through snap fit connections, adhesives, or mechanical fixation devices such as pins or screws. In some embodiments, handle 28 is manufactured as an integral portion of housing 26.

Shaft 34 of catheter 22 may be fixed into a channel of housing 26 or locked in place for a treatment session. Catheter 22 may be produced in different lengths or diameters with different configurations of needles or tip 36. A clinician may be able to interchange catheter 22 with housing 26. In other embodiments, catheter 22 may be manufactured within housing 26 such that catheter 22 may not be interchanged.

Shaft 34 is a rigid structure that is manufactured of stainless steel or another metal alloy and insulated with a polymer such as nylon or polyurethane. Alternatively, shaft 34 may be constructed of a rigid polymer or composite material. Shaft 34 includes one or more channels that house the needle and a cystoscope. Tip 36 may be constructed of an optically clear polymer such that the clinician may view the urethra during catheter 22 insertion. Shaft 34 and tip 36 may be attached with a screw mechanism, snap fit, or adhesives. Tip 36 also includes openings that allow the needle to exit catheter 22 and extend into prostate 24.

In some embodiments, housing 26, handle 28 of housing 26, or trigger 30 may include dials or switches to control the deployment of the needle. These controls may finely tune the ability of the clinician to tailor the therapy for patient 12. Housing 26 may also include a display that shows the clinician the tissue property measured to verify the outcome of the ablation therapy. For example, the temperature detected by the needle may be displayed directly on therapy device 20 for easy viewing.

In some embodiments, shaft 34 and tip 36 may be configured to house two or more needles. For example, multiple needles may be employed to treat a larger volume of tissue at one time and/or provide more accurate feedback relating to the outcome of the ablation therapy.

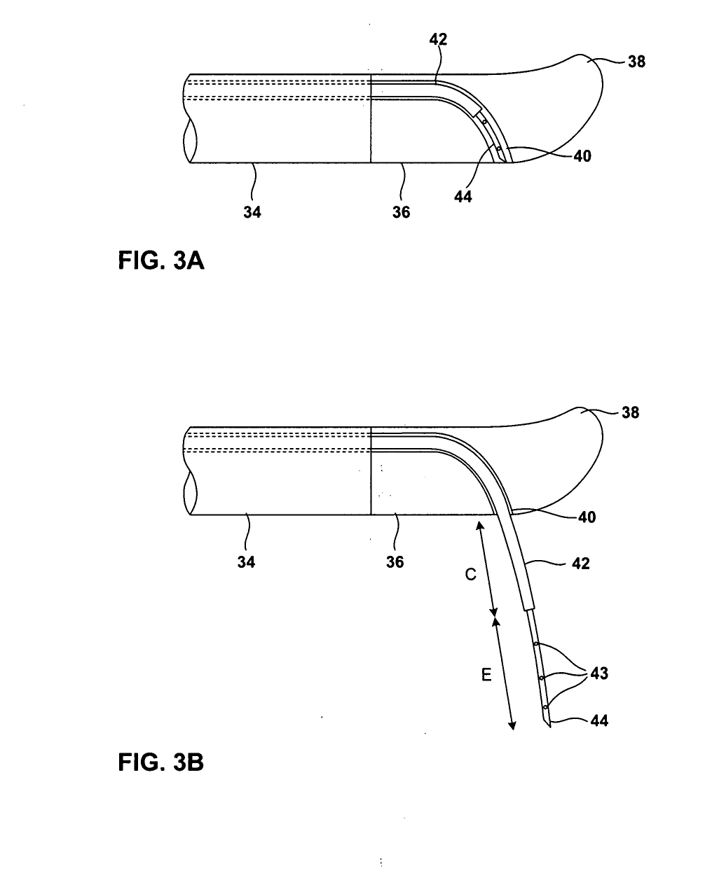

FIGS. 3A and 3B are cross-sectional side views of an exemplary catheter tip from which a therapy needle exits to reach the target tissue. As shown in FIG. 3A, shaft 34 is coupled to tip 36 at the distal end of catheter 22. Tip 36 includes protrusion 38 that aids in catheter insertion through the urethra. Tip 36 also includes channel 40 which allows needle 44 to exit tip 36. Needle 44 is insulated with, sheath 42, such that the exposed portion of needle 44 may act as an electrode. A portion of needle 44 may also sense a tissue property to provide feedback regarding the outcome of the ablation therapy.

Channel 40 continues from tip 36 through shaft 34. The curved portion of channel 40 in tip 36 deflects needle 44 such that the first needle penetrates the target tissue from the side of catheter 22. The curvature of channel 40 may be altered to produce different entry angles of needle 44. Needle 44 may not extend beyond the distal end of tip 36. In other words, needle 44 may exit at or near the side of catheter 22, wherein the side is a lengthwise edge substantially facing the wall of the urethra. The wall of the urethra is a tissue barrier as it surrounds catheter 22. In some embodiments, the distal end of needle 44 may stop at a point further from housing 26 than the distal end of tip 36.

As shown in FIG. 3B, needle 44 has been deployed from tip 36 of catheter 22. The exposed length E of needle 44 may be varied by controlling the position of sheath 42. The covered length C of needle 44 is that length of the first needle outside of tip 36 that is not delivering energy to the surrounding tissue. Exposed length E may be controlled by the clinician to be generally between 1 mm and 50 mm. More specifically, exposed length E may be between 6 mm and 16 mm. Covered length C may be generally between 1 mm and 50 mm. Specifically, covered length C may also be between 5 mm and 7 mm. Once needle 44 is deployed, needle 44 may be locked into place until the ablation therapy is completed.

In some embodiments, needle 44 is a hollow needle which allows conductive fluid, i.e., saline, to flow from generator 14 to the target tissue. Needle 44 may include multiple holes 43 which allow the conductive fluid to flow into the target tissue and increase the effective size of the needle electrode since the conductive fluid may help deliver RF energy to the target tissue. The conductive fluid may also more evenly distribute the RF energy to the tissue to create more uniform lesions. In some embodiments, needle 44 may also include a hole at the distal tip of needle 44. In other embodiments, needle 44 may only include a hole at its distal tip. Generator 14 may include a pump that delivers the conductive fluid.

Alternatively, needle 44 may not deliver a conductive fluid to the target tissue. In this case, needle 44 may be solid or hollow and act as a dry electrode. Delivering energy through needle 44 without a conductive fluid may simplify the ablation procedure and reduce the cost of ablation therapy.

Needle 44 may be used to measure a tissue property to obtain feedback regarding the outcome of the ablation therapy. For example, a portion of needle 44 may be used to measure tissue temperature or tissue impedance after ablation therapy is stopped. In some embodiments, tissue impedance is measured between needle 44 and a return electrode on the back of patient 12 (e.g., ground pad 23 of FIG. 1). Tissue impedance may be used to determine the volume of the lesion formed by tissue ablation. Generally, the larger the lesion, the higher the tissue impedance. In some embodiments, a correlation between tissue impedance values and lesion size may be determined based on the tissue type and location of the target tissue.

As previously mentioned, in some embodiments, needle 44 may measure tissue temperature. For example, needle 44 may measure the decay of tissue temperature following ablation therapy. Measuring tissue temperature over time may help characterize the size of lesion formed and/or other tissue properties. Since ablated tissue is generally a better insulator than healthy tissue, the temperature of a large lesion may decay more slowly than the temperature of a small lesion upon completion of ablation therapy.

In other embodiments, measuring impedance over time may help characterize the size of lesion formed and/or other tissue properties. Tissue impedance may change as the temperature decays following ablation therapy. In this manner, tissue impedance may provide an indirect measurement of temperature. Also, measuring impedance over time may aid in determining the volume of the lesion formed by tissue ablation. In this manner, the rate of change of tissue impedance may be used in combination with the amplitude of tissue impedance to determine the volume of the lesion formed by tissue ablation.

Needle 44 may remain at the ablation site and measure the tissue property at the ablation site after ablation therapy is completed. In other embodiments, needle 44 may additionally or alternatively measure the tissue property at other sites. For example, needle 44 may be retracted, repositioned, and redeployed to measure a tissue property at a distance from the ablation site.

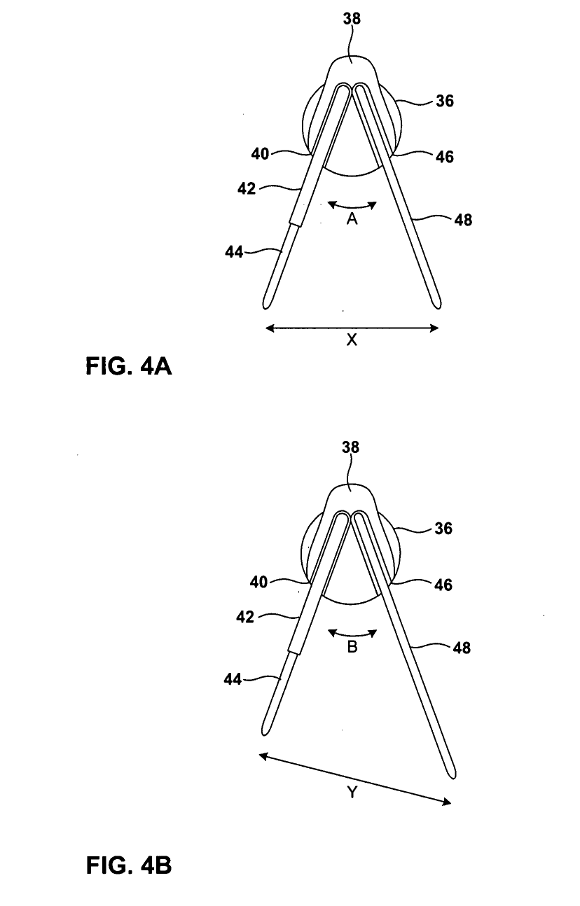

As previously mentioned, multiple needles may be employed to treat a larger volume of tissue at one time and/or provide more accurate feedback relating to the outcome of the ablation therapy. FIGS. 4A and 4B are cross-sectional front views of an example catheter tip 36 and exiting needles 44 and 48. As shown in FIG. 4A, first needle 44 and second needle 48 are deployed from tip 36 of catheter 22. First needle 44 is partially covered by sheath 42 and housed within channel 40. Second needle 48 is housed within channel 46 which mirrors the path of channel 40 shown in FIGS. 3A and 3B. Channels 40 and 46 may or may not be identical in diameter. In the embodiment illustrated in FIG. 4A, first needle 44 and second needle 48 are deployed simultaneously and to the same extended length.

First needle 44 and second needle 48 may be constructed of similar materials or different materials. Exemplary materials may include stainless steel, nitinol, copper, silver, or an alloy including multiple metals. In any case, each needle may be flexible and conduct electricity to promote ablation and/or tissue property detection mechanisms. One or more of needles 44 and 48 may be hollow to include sensors or be formed around such sensors.

Second needle 48 may be a detecting or sensing needle that is used for providing feedback regarding the outcome of the ablation therapy. The tissue property detected by second needle 48 may be impedance, temperature, or another parameter indicative of a lesion produced by tissue ablation. Temperature may be detected by a sensor, such as a thermocouple or thermistor, housed within second needle 48. Additional temperature measurements may be provided by multiple sensors in second needle 48 or even one or more sensors within first needle 44. Generator 14 may measure the signal produced by the sensor and output a measured temperature of the tissue. Impedance may be detected by a measurement between first needle 44 and second needle 48, or second needle 48 and a return electrode located on the back of patient 12 (e.g., ground pad 12 of FIG. 1).

First needle 44 may be used for delivering therapy to a target tissue and second needle 48 may be used for sensing a tissue property after ablation therapy is stopped. For example, if energy is delivered to a target tissue from needle 44, needle 48 may measure a tissue property at a specified distance from the ablating needle 44. In some embodiments, second needle 48 is dedicated to sensing and does not deliver energy to the target tissue.

In other embodiments, both first needle 44 and second needle 48 deliver energy to a target tissue. For example, needles 44 and 48 may both deliver energy to the target tissue proximate to needles 44 and 48, and the energy emitted by needles 44 and 48 may be returned via the ground pad 23 (of FIG. 1). As another example, needles 44 and 48 may deliver bipolar stimulation to ablate a target tissue between first needle 44 and second needle 48 and/or proximate to needles 44 and 48, and one or more of needles 44 and 48 may act as the return electrode that receives energy dispersed from one or more of needles 44 and 48.

In some embodiments, both needles 44 and 48 sense a tissue property after ablation therapy is stopped. Measuring a tissue property with both of needles 44 and 48 may provide a more accurate depiction of the lesion formed. As one example, tissue impedance may be measured between needle 44 and ground pad 23 (FIG. 1) and also between needle 48 and ground pad 23 (FIG. 1). A difference in tissue impedance between needle 44 and ground pad 23 and needle 48 and ground pad 23 may be useful in characterizing the volume of the lesion formed by ablation therapy or another therapy result.

First needle 44 and second needle 48 exit tip 36 at angle A with respect to each other. Angle A may be varied by selecting different catheters 22 before the procedure. Generally, angle A is between 0 degrees and 120 degrees. More specifically, angle A is between 35 degrees and 50 degrees. In the preferred embodiment, angle A is approximately 42.5 degrees. While angle A is bisected by the midline of catheter 22, angle A may be offset to either side so that the needles do not form a symmetrical angle to the catheter.

Once deployed, the length of first needle 44, the length of second needle 48, and the value of angle A determine the distance X between the distal ends of each needle. Distance X may be varied such that second needle 48 is positioned at a distance to effectively provide feedback about the outcome of ablation therapy. Generally, distance X is between 1 mm and 100 mm. More specifically, distance X may be between 6 mm and 20 mm. Preferably, distance X is approximately 13 mm. Distance X may be entered into generator 14 to accurately measure the tissue property of interest (e.g., using thermocouples to measure temperature or measuring tissue impedance between needles 44 and 48). Distance X may be useful when measuring the tissue impedance between first needle 44 and second needle 48.

In some embodiments, a sheath similar to sheath 42 may be included around second needle 48. The sheath may expose the desired length of second needle 48 and prevent fluids from entering channel 46.

In the embodiment illustrated in FIG. 4B, first needle 44 and second needle 48 are extended at angle B with respect to each other. However, first needle 44 and second needle 48 have differing extended lengths. Second needle 48 is deployed at a longer length than first needle 44 to create a distance Y between the distal ends of each needle. In other embodiments, first needle 44 may be extended to a distance greater than second needle 48.

First needle 44 and second needle 48 may be deployed simultaneously using trigger 30 of therapy device 20. An internal mechanism may extend second needle 48 at a faster rate or limit the length of first needle 44 before limiting the length of the second needle 48. In either case, the clinician may control the length of each needle. Generator 14 may determine distance Y based upon the angle B and lengths of each needle, or the clinician may input the needle lengths into the generator. In other embodiments, first needle 44 and second needle 48 may have independent triggers 30 or other deployment mechanisms that allows the clinician to utilize two different lengths for the first and second needle. Increasing or decreasing distance Y may allow the clinician to accurately determine the size of a produced lesion.

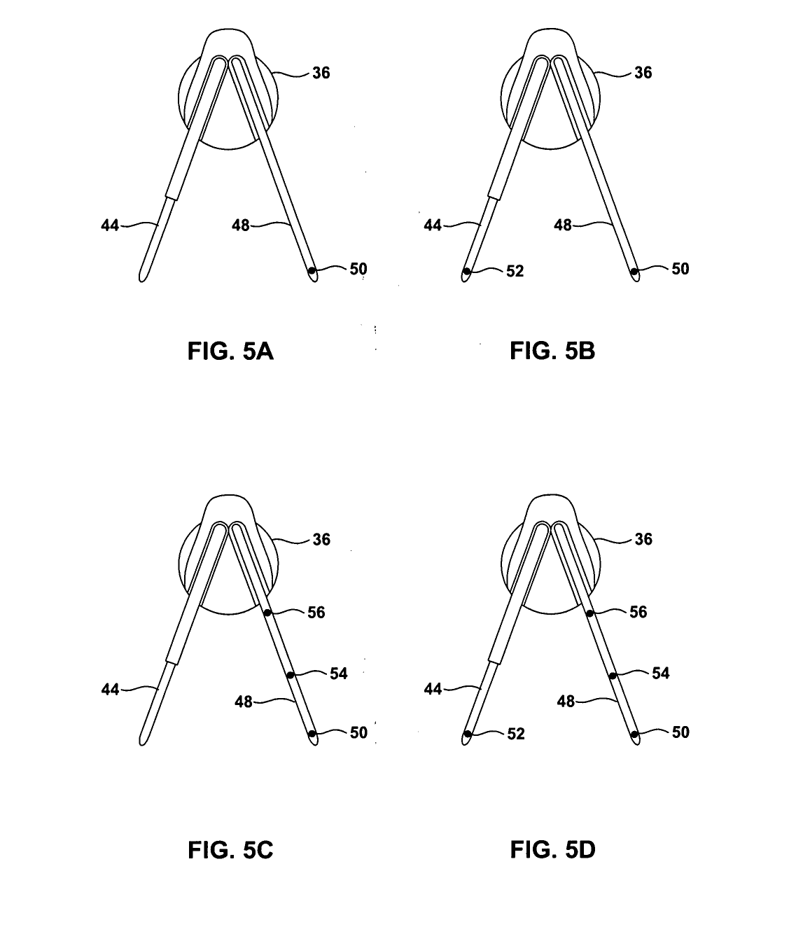

FIGS. 5A-5D are cross-sectional front views of exemplary ablation and sensing needles with varying sensing element configurations. As shown in FIGS. 5A-5D, thermocouples are located at different positions of first needle 44 and second needle 48. These configurations may be available to the clinician by changing therapy device 20 or catheter 22.

FIG. 5A shows thermocouple 50 located at the distal end of second needle 48. As described with respect to FIGS. 4A and 4B, first needle 44 delivers energy to a target tissue. Second needle 48 may, but need not, deliver energy to the target tissue. In some embodiments, needle 48 is dedicated to sensing a tissue property after ablation therapy is stopped. FIG. 5B shows thermocouple 50 at the distal end of second needle 48 and thermocouple 52 located at the distal end of first needle 44. Providing multiple thermocouples to obtain more than one temperature reading may allow a temperature gradient to be monitored between the sensors.

FIG. 5C is an example of three thermocouples 50, 54 and 56 located at various positions on second needle 48. Thermocouples 50, 54 and 56 may provide temperatures for multiple distances away from the source of ablation energy, e.g., first needle 44. FIG. 5D includes thermocouples 50, 54 and 56 on second needle 48 and thermocouple 52 on first needle 44. The configuration of FIG. 5D may allow a more accurate temperature profile of the lesion produced. The clinician may desire more feedback to more accurately determine the volume of the lesion produced or other therapy results. In some embodiments, the temperature readings may be compared to data stored in look-up tables to determine the volume of the lesion produced. In other embodiments, the volume of the lesion formed may be calculated based on the measured temperature readings. The data stored in the look-up tables and/or the formulas used in the calculations may be based on clinical data obtained from other patients.

In other embodiments, more or less thermocouples may be used to detect temperatures at various locations within prostate 24. In addition, sensors may include thermistors, a combination of thermistors and thermocouples, or any other temperature sensing elements. In some embodiments, infrared light or chemical sensors may be provided by second needle 48 to measure the temperature of the target tissue or lesion.

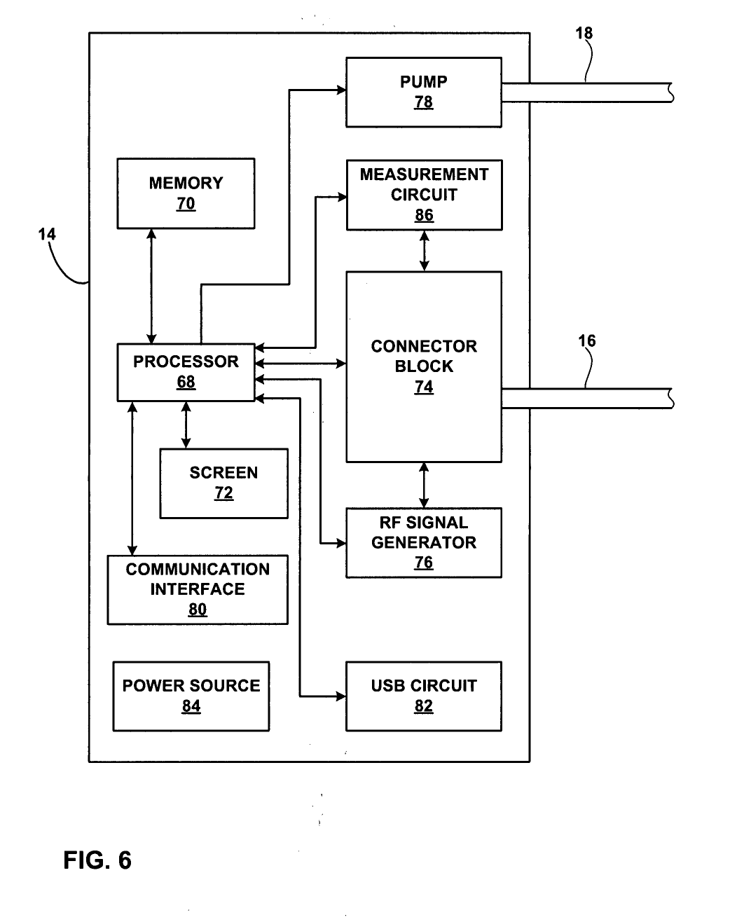

FIG. 6 is functional block diagram illustrating components of an exemplary generator system. In the example of FIG. 6, generator 14 includes a processor 68, memory 70, screen 72, connector block 74, RF signal generator 76, measurement circuit 86, pump 78, telemetry interface 80, USB circuit 82, and power source 84. As shown in FIG. 6, connector block 74 is coupled to cable 16 for delivering RF energy produced by RF signal generator 76 and detecting tissue properties with measurement circuit 86. Pump 78 produces pressure to deliver fluid through tube 18.

Processor 68 controls RF signal generator 76 to deliver RF energy therapy through connector block 74 according to therapy parameter values stored in memory 70. Processor 68 may receive such parameter values from screen 72, telemetry interface 80, or USB circuit 82. When signaled by the clinician, which may be a signal from therapy device 20 conveyed through connector block 74, processor 68 communicates with RF signal generator 76 to produce the appropriate RF energy. As needed, pump 78 provides fluid to irrigate the ablation site or provides fluid to the electrode during wet electrode ablation.

In a preferred embodiment, the RF signal generator may have certain performance parameters. In this exemplary case, the generator may provide RF energy into two channels with a maximum of 50 Watts per channel. The ramp time for a 50 Watt change in power may occur in less than 25 milliseconds. The output power may be selected in 1 Watt steps. The maximum current to be provided to the patient may be 1.5 Amps, and the maximum voltage may be 180 Volts.

Connector block 74 may contain an interface for a plurality of connections, not just the connection for cable 16. These other connections may include one for a return electrode (e.g., ground pad 23 of FIG. 1 or a second needle), a second RF energy channel, or separate tissue property sensors. As mentioned previously, connector block 74 may be a variety of blocks used to diagnose or treat a variety of diseases. All connector blocks may be exchanged and connect to processor 68 for proper operation. Pump 78 may be replaceable by the clinician to allow replacement of a dysfunctional pump or use of another pump capable of pumping fluid at a different flow rate.

Measurement circuit 86 may be configured to measure the impedance between first needle 44 and second needle 48, another impedance measurement, or temperature measurements from one or more sensors located in second needle 48 and/or first needle 44. In some embodiments, measurement circuit 86 may perform multiple sensing calculations to provide the clinician with impedance and temperature measurements.

Tissue properties, such as temperature measurements or impedance measurements, may also be monitored with measurement circuit 86 or processor 68 to provide an indicator of the therapy outcome. For example, the decay of tissue temperature following ablation therapy (e.g., after energy delivery is stopped) may be measured to help characterize the size of lesion formed and/or other tissue properties. Since ablated tissue is generally a better insulator than healthy tissue, the temperature of a large lesion may decay more slowly than the temperature of a small lesion upon completion of ablation therapy. In other embodiments, impedance may be measured over time. Tissue impedance may change as temperature decays, providing an indirect measurement of temperature. The rate of impedance change may also be used to aid in determining the volume of the lesion formed by ablation therapy. In other embodiments, changes to other tissue properties may be tracked over time.

Measurement circuit 86 may also perform calibration procedures to ensure accurate measurements of the tissue properties. The calibration of sensing elements may occur before every ablation treatment, during treatment, after every treatment, when generator 14 is turned on, or at any time the clinician desires to calibrate the sensors.

Processor 68 may also control data flow from the therapy. Data such as RF energy produced, tissue properties measured from measurement circuit 86, and fluid flow may be channeled into memory 70 for later analysis. Processor 68 may comprise any one or more of a microprocessor, digital signal processor (DSP), application specific integrated circuit (ASIC), field-programmable gate array (FPGA), or other digital logic circuitry. Memory 70 may include multiple memories for storing a variety of data. For example, one memory may contain therapy parameters, one may contain generator operational files, and one may contain measured therapy data. Memory 70 may include any one or more of a random access memory (RAM), read-only memory (ROM), electronically-erasable programmable ROM (EEPROM), flash memory, or the like.

Processor 68 may also send data to USB circuit 82 when a USB device is present to save data from therapy. USB circuit 82 may control both USB ports in the present embodiment; however, USB circuit 82 may control any number of USB ports included in generator 14. In some embodiments, USB circuit may be an IEEE circuit when IEEE ports are used as a means for transferring data.

The USB circuit may control a variety of external devices. In some embodiments, a keyboard or mouse may be connected via a USB port for system control. In other embodiments, a printer may be attached via a USB port to create hard copies of patient data or summarize the therapy. Other types of connectivity may be available through the USB circuit 82, such as internet access.

Communications with generator 14 may be accomplished by radio frequency (RF) communication or local area network (LAN) with another computing device or network access point. This communication is possible through the use of communication interface 80. Communication interface 80 may be configured to conduct wireless or wired data transactions simultaneously as needed by the clinician.

Generator 14 may communicate with a variety of devices to enable appropriate operation. For example, generator 14 may utilize communication interface 80 to monitor inventory, order disposable parts for therapy from a vendor, and download upgraded software for a therapy. For example, generator 14 may order a new catheter 22. In some embodiments, the clinician may communicate with a help-desk, either computer directed or human staffed, in real-time to solve operational problems quickly. These problems with generator 14 or a connected therapy device may be diagnosed remotely and remedied via a software patch in some cases.

Screen 72 is the interface between generator 14 and the clinician. Processor 68 controls the graphics displayed on screen 72 and identifies when the clinician presses on certain portions of screen 72, which is sensitive to touch control. In this manner, screen 72 operation may be central to the operation of generator 14 and appropriate therapy or diagnosis. Screen 72 may also display measured tissue property values.

Processor 68 may analyze data received from measurement circuit 86 and provide the results of the analysis to the clinician via screen 72. For example, processor 68 may analyze the measured tissue property received from measurement circuit 86 and provide an indicator of the therapy outcome based on the analysis. In some embodiments, processor 68 determines a volume of the lesion formed via ablation therapy based on the measured tissue properties and displays the determined volume on screen 72. In other embodiments, processor 68 determines a condition of the tissue at the site of the measurement (e.g., not ablated, partially ablated, fully ablated, over ablated, etc) or another indicator of the therapy outcome. In some embodiments, processor 68 may compare the measured tissue property received from measurement circuit 86 to data stored in look-up tables and provide the indicator of the therapy outcome based on the comparison. In other embodiments, processor 68 may calculate the indicator of therapy outcome (e.g., the volume of the lesion formed) by inputting the measured tissue property into one or more formulas. The formulas used in the calculations and/or the data stored in the look-up tables may be based on clinical data obtained from other patients.

Power source 84 delivers operating power to the components of generator 14. Power source 84 may utilize electricity from a standard 115 Volt electrical outlet or include a battery and a power generation circuit to produce the operating power. In some embodiments, the battery may be rechargeable to allow extended operation. Recharging may be accomplished through the 115 Volt electrical outlet. In other embodiments, traditional batteries may be used.

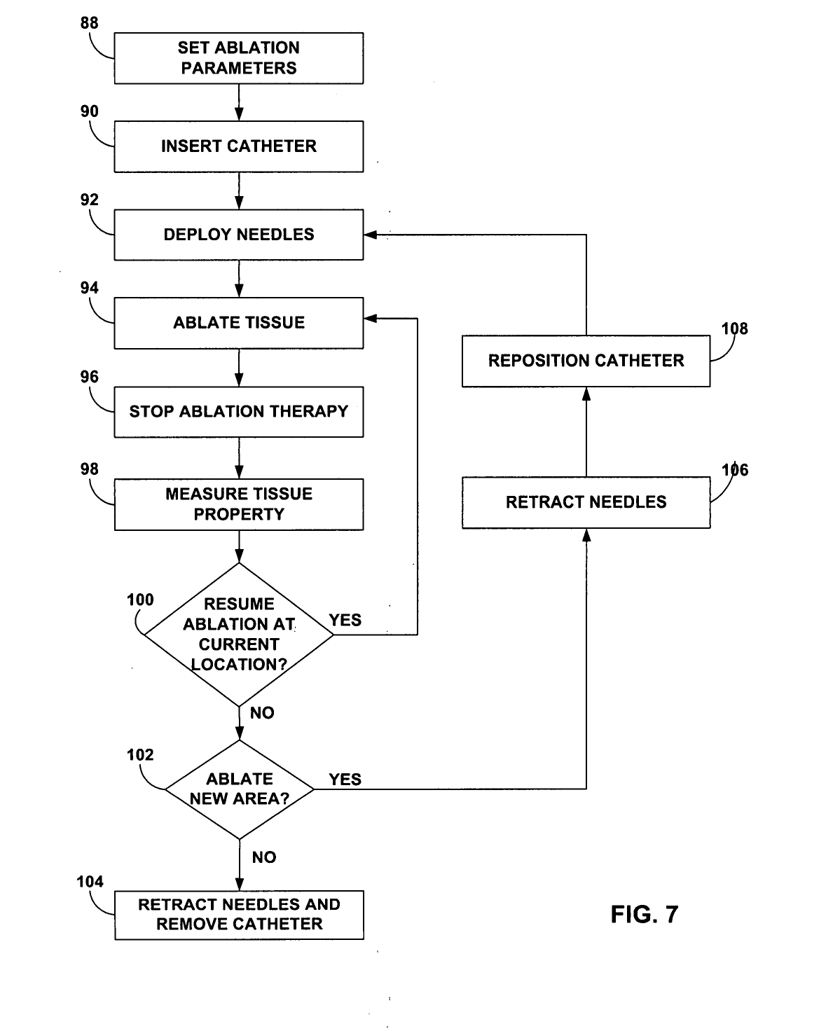

FIG. 7 is a flow diagram illustrating an example technique for verifying the outcome of a tissue ablation procedure. The clinician sets ablation parameters in generator 14 (88). Ablation parameters may include RF power, needle lengths, or other parameters related to the therapy. Selecting a desired catheter 22 configuration may be an ablation parameter as well. The clinician next inserts catheter 22 into the urethra of patient 12 until tip 36 is correctly positioned adjacent to prostate 24 (90). The clinician may use a cystoscope within catheter 22 to guide the catheter. Once correctly positioned, the clinician deploys first needle 44 and second needle 48 into prostate 24 (92).

The clinician starts tissue ablation by pressing a button on generator 14 or therapy device 20 (94). Conductive fluid may or may not be delivered by one or more of first needle 44 and second needle 48. When deemed appropriate, the clinician stops ablation (96). For example, the clinician may choose a treatment time based on experience to achieve a desired therapy outcome. A tissue property is measured (98) to help evaluate the outcome of the therapy. As previously described, the measured tissue property may be temperature, impedance, or any other appropriate tissue property. The measured tissue property may provide an indication of the therapy outcome, such as a volume of lesion formed.

The measured tissue property may provide an indication as to whether or not a desired therapy result has been achieved (100). If the measured tissue property provides an indication that a desired therapy result has not been achieved, the clinician may chose to resume ablation therapy at the same location (94). If the clinician is satisfied with the therapy result at the current location, the clinician may decide whether or not to ablate a new area of prostate 24 (102).

If the clinician does not want to ablate a new area of prostate 24 (102), the clinician retracts needles 44 and 48 and removes catheter 22 from patient 12 (104). If the clinician desires to ablate more tissue, the clinician retracts needles 44 and 48 (106), repositions catheter 22 adjacent to the new tissue area (108), and deploys the needles once more (92). Ablation may begin again to treat more tissue (94).

In some embodiments, the tissue property measurement is taken at the site of the ablation. Additionally or alternatively, a tissue property measurement may be taken at another location. For example, a clinician may initially measure a tissue property at the site of tissue ablation. If the tissue appears to be ablated at the location of the measurement, the clinician may retract, reposition, and redeploy the one or more needles at a second location to detect a tissue property at that location. In some embodiments, the clinician may take a series of probing measurements in different areas of the prostate to verify lesion formation. For example, the clinician may wish to verify lesion formation in specific areas of the prostate, such as areas that have alpha-receptors or a high density of nerve fibers.

The preceding specific embodiments are illustrative of the practice of the invention. It is to be understood, therefore, that other expedients known to those skilled in the art or disclosed herein may be employed without departing from the invention or the scope of the claims.

Various embodiments of the invention have been described. These and other embodiments are within the scope of the following claims.

1. A method for providing feedback regarding the results of tissue ablation, the method comprising:
deploying one or more needles from a catheter into a target tissue;
delivering energy via at least one of the one or more needles to ablate at least a portion of the target tissue to form a lesion;
stopping energy delivery via the at least one of the one or more needles; and
measuring a tissue property via at least one of the one or more needles after the energy delivery has been stopped.

2. The method of claim 1, further comprising determining a volume of the lesion using the measured tissue property.

3. The method of claim 1, wherein the one or more needles comprise a needle that measures the tissue property and delivers energy to the target tissue.

4. The method of claim 1, wherein the one or more needles comprise a first needle that delivers energy to the target tissue and a second needle that measures the tissue property.

5. The method of claim 4, wherein the second needle delivers energy to the target tissue.

6. The method of claim 5, wherein the target tissue is at least one of between or proximate to the first and second needles, and wherein delivering energy to the target tissue comprises delivering energy to the target tissue using bipolar ablation.

7. The method of claim 1, further comprising delivering a conductive fluid to the target tissue via at least one of the one or more needles.

8. The method of claim 7, further comprising moving the fluid through a plurality of holes in at least one of the one or more needles.

9. The method of claim 1, wherein the tissue property is temperature.

10. The method of claim 9, wherein the one or more needles comprise a first needle and a second needle, further comprising measuring a temperature difference between the first needle and the second needle.

11. The method of claim 9, further comprising measuring a temperature change over time.

12. The method of claim 1, wherein the tissue property is impedance.

13. The method of claim 1, further comprising displaying the tissue property measurement to a user.

14. The method of claim 1, wherein delivering energy via the at least one of the one or more needles is at least partially controlled by an insulated sleeve covering a portion of the at least one of the one or more needles.

15. The method of claim 1, wherein the target tissue is a prostate.

16. The method of claim 1, further comprising retracting the needle after at least a portion of the target tissue is ablated.

17. A system comprising:
a generator that generates energy to ablate at least a portion of a target tissue to form a lesion;
one or more needles that deliver the energy to the target tissue, wherein at least one of the needles comprises a measurement device that measures a tissue property of the target tissue after the lesion is formed; and
a processor that analyzes the measured tissue property and provides an indicator of therapy outcome based on the measured tissue property.

18. The system of claim 17, further comprising a display, wherein the indicator of therapy outcome is displayed on the display.

19. The system of claim 17, further comprising a pump to deliver a conductive fluid to the target tissue via at least one of the one or more needles.

20. The system of claim 19, wherein the at least one of the one or more needles comprises a plurality of holes that deliver the conductive fluid to the target tissue.

21. The system of claim 17, wherein the target tissue is a prostate.

22. The system of claim 17, wherein the indicator of therapy outcome comprises a volume of the lesion.

23. The system of claim 17, wherein the tissue property is temperature.

24. The system of claim 23, wherein the one or more needles comprise a first needle and a second needle, further comprising measuring a temperature difference between the first needle and the second needle.

25. The system of claim 23, wherein a change in temperature is measured over time.

26. The system of claim 17, wherein the tissue property is impedance.

27. The system of claim 26, wherein a change in impedance is measured over time.

28. The system of claim 17, further comprising a return electrode pad that receives energy dispersed from the one or more needles.

29. The system of claim 28, wherein the one or more needles comprise a first needle and second needle, further comprising measuring an impedance difference between the first needle and the return electrode pad and the second needle and the return electrode pad.

30. The system of claim 17, further comprising a catheter that houses at least a portion of the one or more needles.

31. A computer-readable medium comprising instructions for causing a programmable processor to:
deliver energy via one or more needles to ablate at least a portion of a target tissue to form a lesion;
receive a tissue property measurement, wherein the tissue property measurement is measured via at least one of the one or more needles after the energy delivery has been stopped; and
analyze the measured tissue property and provide an indicator of therapy outcome based on the measured tissue property.

32. The computer-readable medium of claim 31, wherein the indicator of therapy outcome comprises a volume of the lesion.
